(19) United States
(12) Patent Application Publication
(10) Pub. No.: US 2018/0042875 A1
(43) Pub. Date: Feb. 15, 2018

(54) COMPOUND USE IN PROMOTING ENERGY EXPENDITURE

(71) Applicant: NESTEC S.A., Vevey (CH)

(72) Inventors: Denis Marcel Barron, Lutry (CH); Carles Canto Alvarez, Cuarnens (CH); Sameer Kulkarni, Lausanne (CH); Joanna Ratajczak, Lausanne (CH)

(21) Appl. No.: 15/554,855

(22) PCT Filed: Feb. 29, 2016
(86) PCT No.: PCT/EP2016/054188

§ 371 Date: Aug. 31, 2017

(30) Foreign Application Priority Data
Mar. 3, 2015 (EP) 15157349.0

(51) Int. Cl. A61K 31/191; A23F 3/16; A23L 33/00; A23L 33/105

(52) U.S. Cl. CPC A61K 31/191; A23L 33/105; A23F 3/16; A23L 33/30; A23L 33/40

(57) ABSTRACT

The present invention provides a compound of structural formula (1), a salt thereof for use in promoting energy expenditure and/or thermogenesis.

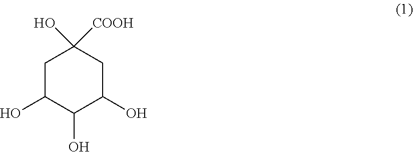 [SMILES: O=C(O)C1(O)CC(O)C(O)C(O)C1]

### FIELD OF INVENTION

The present invention provides a compound for use in promoting energy expenditure and/or thermogenesis. The invention also relates to a compound for use in attaining or maintaining weight loss.

### BACKGROUND

Obesity is a chronic metabolic disorder that has reached epidemic proportions in many areas of the world and is the major risk factor for serious co-morbidities such as type 2 diabetes mellitus, cardiovascular disease, dyslipidaemia and certain types of cancer (World Health Organ Tech Rep Ser. 2000; 894:i-xii, 1-253).

Obesity refers to a condition in which an individual weighs more than usual as a result of excessive accumulation of energy intake from carbohydrate, fat, and the like in the form of fat under the skin or around the viscera.

Empirical data suggests that a weight loss of at least 10% of the initial weight results in a considerable decrease in risk for obesity related co-morbidities (World Health Organ Tech Rep Ser. 2000; 894:i-xii, 1-253). However, the capacity to lose weight shows large inter-subject variability.

Because obesity is induced when the amount of energy intake exceeds the amount of energy consumed, in order to ameliorate obesity, a method of decreasing the amount of energy intake from fat, carbohydrate, and the like or a method for increasing the amount of energy consumption by promoting in vivo metabolism is desired. Therefore, improvements of dietary habit and exercise are considered as an effective method for the prevention and amelioration of obesity and obesity related disclosures.

Exercise activates energy metabolism mainly in muscle and increases energy consumption, and thus is an effective method for the prevention and amelioration of obesity. However, it is often practically difficult to perform regular exercise, not least due to time constraints.

There is a lot of interest in the health benefits, including weight loss properties, of natural products such as plant species. Chlorogenic acids are esters of hydroxycinnamic acids such as caffeic, ferulic, and/or p-coumaric with quinic acid, and are common in nature and in a number of dietary sources (Clifford et al., J. Sci. Food Agric. 1999, 79, 362-372; Clifford et al., J. Sci. Food Agric. 2000, 80, 1033-1043). The effects of chlorogenic acids on weight management have been linked to a decrease in oxidative stress and it has been hypothesized that such effects are related to chlorogenic acid molecules.

Since it is known that the overproduction of free radicals like peroxynitrite may contribute to several diseases including obesity, a number of extracts known to be rich in chlorogenic acids have been investigated for their radical scavenging effects. These include extracts of Salicorna herbacea (Hwang et al., Chem.-Biol. Interact. 2009, 181, 366-376) and of Korean mountainous vegetables (Nugroho et al., Arch. Pharmacal Res. 2009, 32, 1361-1367; Nugroho et al., Nat. Prod. Sci. 2010, 16, 80-87).

Mate is a popular beverage in South America and its anti-obesity effects have been studied. A recent study has focussed on the effects of mate on GLP-1 levels with particular attention directed to chlorogenic acids and matesaponins, based on the fact that they are the major constituents of the beverage (Hussein et al., Biol. Pharm. Bull. 2011, 34, 1849-185). In a study aimed at investigating the anti-obesity effects of an extract of the fruit of Mulberry (Morus alba), focus was again made on phenols and polyphenols which were the only constituents analysed in the extract (Peng et al., J. Agric. Food Chem. 2011, 59, 2663-2671).

Dandelion has been suggested to prevent metabolic disorders. It has been recently demonstrated that dandelion extract inhibited adipogenesis and lipid metabolism in 3T3-L1 adipocytes. Focus was again made on the main phenolic compounds of the extract, which included caffeic and chlorogenic acid, in accordance with previous studies having shown that these two compounds inhibited adipogenesis in 3T3-L1 cells (Gonzalez-Castejon et al., Phytother. Res. 2014, 28, 745-752).

Chellan et al. 2012 assessed the effects of an aqueous extract of Athrixia phylicoides on glucose metabolism in vitro. Based on the abundant literature related to the benefits of plant phenolic compounds on type-2 diabetes and obesity, only the phenolic profile of the extract was analysed. These phenolic compounds including a number of chlorogenic acids were postulated to play a role in the activity of the extract (Chellan et al., Phytomedicine 2012, 19, 730-736).

Glucose uptake can be decreased by the use of inhibitors of α-amylase that delay postprandial polysaccharide digestion. A number of natural phenolic compounds are inhibitors of α-amylase, and chlorogenic acids also display this property. The fact that quinic acid was a poor inhibitor as compared to caffeic and chlorogenic acids demonstrated that the inhibition was associated with the phenolic part of chlorogenic acids (Narita et al., J. Agric. Food Chem. 2009, 57, 9218-9225). Another study confirmed that quinic acid had no effect on the enzymes involved in the digestion of carbohydrates (maltase, sucrase) and lipids (lipase). In contrast, the phenolic acid esters of quinic acid present in coffee, especially the di-caffeoylquinic acid esters, displayed inhibitory activities towards these digestive enzymes.

Svetol®, a proprietary green coffee decaffeinated extracts which is rich and standardized in chlorogenic acids, has been developed by Naturex SA. Svetol® has been demonstrated to induce weight loss in overweight volunteers (Dellalibera et al., Phytotherapie 2006, 4, 194-197). The mechanism was suggested to involve glucose metabolism, (Blum et al., Nutrafoods 2007, 6, 13-1) itself related to the inhibition of hepatic glucose-6-phosphatase by chlorogenic acids (Henry-Vitrac et al., J. Agric. Food Chem. 2010, 58, 4141-4144; Schindler et al., Drug Dev. Res. 1998, 44, 34-40; Hemmerle et al., J. Med. Chem. 1997, 40, 137-145; Arion et al., Arch. Biochem. Biophys. 1997, 339, 315-32). Thom investigated the effects of Coffee Slender®, an instant coffee enriched with Svetol® on glucose absorption. The significant decrease in body weight and fat percentage of Coffee Slender® as compared to regular instant coffee was attributed again to the chlorogenic acids brought by the Svetol® extract ((Thom, J. Int. Med. Res. 2007, 35, 900-908).

The thermogenesis and lipolysis effects of coffee have been largely attributed to caffeine (Greenberg et al., Am. J. Clin. Nutr. 2006, 84, 682-693; George et al., Crit. Rev. Food Sci. Nutr. 2008, 48, 464-486). Among the non-caffeine representatives of coffee, the chlorogenic acids, due to their phenolic character, have received most of the attention. In an effort to understand the effects of coffee components on human energy metabolism, the phenolic fraction of coffee, rich in chlorogenic acids was studied. A 4 weeks ingestion of the chlorogenic acid beverage led to a significantly higher postprandial energy expenditure than the control beverage (Soga et al., Biosci., Biotechnol., Biochem. 2013, 77, 1633-1636).

The consumption of a coffee phenolic fraction in which the amount of caffeoylquinic and feruloylquinic acids have been quantified was shown to increase fat utilization in humans (Ota et al., J. Health Sci. 2010, 56, 745-751). Murase et al. (2011) have demonstrated that the phenolic fraction of roasted coffee (mostly caffeoylquinic and feruloylquinic esters) enhanced the energy metabolism and reduced lipogenesis in C57BL/6J mice (Murase et al., Am. J. Physiol. 2011, 300, E122-E133).

Thus, while chlorogenic acid has been widely studied, the primary focus has been on the phenolic component of the molecule. In contrast, little attention has been paid to the quinic acid component of the molecule.

### SUMMARY OF THE INVENTION

We have surprisingly found that quinic acid was able to increase oxygen consumption and carbohydrate metabolism in mice on a chow diet. Quinic acid was also found to reduce body weight gain and reduce fat deposition, improve stamina and improve exercise effect.

Thus, in a first aspect the present invention provides a compound of structural formula 1:

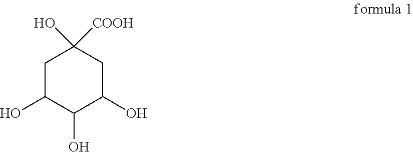 [SMILES: O=C(O)C1(O)CC(O)C(O)C(O)C1]

or a salt thereof for use in promoting energy expenditure and/or thermogenesis.

The compound for use according to the first aspect of the invention may be for use in promoting carbohydrate burning.

The compound for use according to the first aspect of the invention may be for use in attaining or maintaining weight loss in a subject.

The compound for use according to the first aspect of the invention may be for use in the treatment or prevention of obesity or an obesity related disorder.

The compound may be used to reduce fat mass and substantially maintain lean mass.

The compound for use according to the first aspect of the invention may be for use in maintaining body temperature in a subject.

The compound for use according to the first aspect of the invention may be for use in improving stamina in a subject.

The compound for use according to the first aspect of the invention may be for use in improving an exercise effect.

The compound for use according to the first aspect of the invention may have the structural formula 1a:

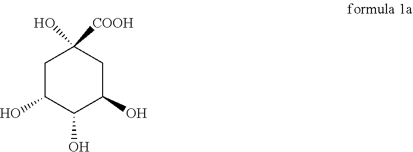 [SMILES: O=C(O)[C@]1(O)C[C@@H](O)[C@@H](O)[C@H](O)C1]

In a further aspect the present invention provides a composition comprising a compound of formula 1 or 1a as an active agent for use as defined herein.

The composition may be selected from the group consisting of a food product, a food extract, drink, food additive, nutritional supplement, medical food, pet food product and a powdered nutritional formulation to be reconstituted in milk or water.

The present invention further provides a pharmaceutical or nutraceutical composition comprising a compound of formula 1 or 1a.

The present invention further provides a food or food extract enriched with a compound of formula 1 or 1a.

The composition or food extract may be derived from coffee, sea buckthorn, bilberry, whortleberry, blueberry, kiwifruit, tamarillo, prune, crowberry, cranberry, peach, apple, sunflower, mayhaw, tea, grapes, black current, medlar, babaco, blackberry, bartlett pear, orange, lemon, citrus, cocoa, quince, chicory, feijoa, pear, sweet potato, japanese persimmon, tomato, banana, pineapple, olive, cherry, pepino, prickly pear, fenugreek, bitter melon or red chicory.

The food extract may be derived from a fermented product. For example, the fermented product may be vinegar or tea.

In a further aspect the present invention provides a diet product for use as part of a low calorie diet for weight loss, wherein the diet product comprises a compound of formula 1 or 1a.

In one aspect the present invention provides the use of a compound of formula 1 or 1a for promoting energy expenditure, promoting thermogenesis, attaining or maintaining weight loss, treatment or prevention of obesity or an obesity related disorder, improving stamina and/or improving an exercise effect.

In another aspect the present invention provides a compound of formula 1 or 1a for use in promoting energy expenditure, promoting thermogenesis, attaining or maintaining weight loss, treatment or prevention of obesity or an obesity related disorder, improving stamina and/or improving an exercise effect.

In another aspect the present invention provides the use of a compound of formula 1 or 1a in the preparation of a product for promoting energy expenditure, promoting thermogenesis, attaining or maintaining weight loss, treatment or prevention of obesity or an obesity related disorder, improving stamina and/or improving an exercise effect.

In another aspect the present invention provides the use of a compound of formula 1 or 1a in the preparation of a diet product.

In a further aspect the present invention relates to a method for promoting energy expenditure, promoting thermogenesis, attaining or maintaining weight loss, treatment or prevention of obesity or an obesity related disorder, improving stamina and/or improving an exercise effect comprising administering compound of formula 1 or 1a to a subject.

### DESCRIPTION OF THE FIGURES

FIG. 1. Quinic acid is tolerated at both low and high doses in mice fed on a chow diet. Graph shows the average of 6 independent measures during 6 different weeks. Results are expressed as mean+/−SEM of n=8-10 mice per group. * indicates statistical significant difference vs. Vehicle group at P<0.05

FIG. 2. Quinic acid is tolerated at both low and high doses in mice fed on a high fat diet. Graph shows the average of 6 independent measures during 6 different weeks. Results are expressed as mean+/−SEM of n=8-10 mice per group.

FIG. 3. Quinic acid prevents body weight gain either in low or high-fat diet. Results are expressed as mean+/−SEM of n=8-10 mice per group. * indicates statistical significant difference vs. respective Veh treated group at P<0.05

FIG. 4. Quinic acid prevents fat deposition. Results are expressed as mean+/−SEM of n=8-10 mice per group. * indicates statistical significant difference vs. respective Vehicle group at P<0.05.

FIG. 5. Quinic acid does not alter daily activity in mice. Results are expressed as mean+/−SEM of n=8-10 mice per group.

FIG. 6. Quinic acid enhances oxygen consumption and carbohydrate metabolism during the dark phase. Results are expressed as mean+/−SEM of n=8-10 mice per group. * indicates statistical significant difference vs. Vehicle group at P<0.05. The total carbohydrate oxidation was calculated based using the equation:

carbohydrate oxidation=(4.55*VO2−3.21*VCO2) where VO2 is volume of oxygen consumed and VCO2 is volume of CO2 produced.

FIG. 7. Quinic acid modestly enhances glucose tolerance. Results are expressed as mean+/−SEM of n=8-10 mice per group. * indicates statistical significant difference vs. Vehicle group at P<0.05

FIG. 8. Quinic acid does not alter insulin response in chow-fed mice. Results are expressed as mean+/−SEM of n=8-10 mice per group.

FIG. 9. Quinic acid lowers basal glycemia but does not significantly affect insulin response on high-fat fed mice. Results are expressed as mean+/−SEM of n=8-10 mice per group. * indicates statistical significant difference vs. Vehicle group at P<0.05

FIG. 10. Quinic acid enhances thermogenic function. Results are expressed as mean+/−SEM of n=8-10 mice per group. * indicates statistical significant difference vs. Vehicle group at P<0.05

FIG. 11. Quinic acid potentiates endurance performance on high-fat fed mice. Results are expressed as mean+/−SEM of n=8-10 mice per group. * indicates statistical significant difference vs. Vehicle group at P<0.05

### DETAILED DESCRIPTION OF THE INVENTION

Quinic Acid

Quinic acid has the general formula C₇H₁₂O₆.

As used herein, quinic acid refers to a compound of structural formula 1:

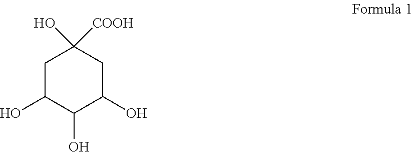 [SMILES: O=C(O)C1(O)CC(O)C(O)C(O)C1]

The quinic acid may be a compound of structural formula 1a:

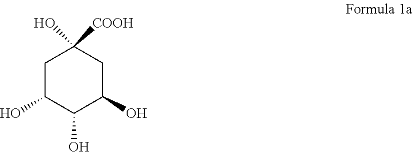 [SMILES: O=C(O)[C@]1(O)C[C@@H](O)[C@@H](O)[C@H](O)C1]

Examples of a salt of quinic acids include a salt with an alkali metal such as sodium and potassium, a salt with an alkaline earth metal such as magnesium and calcium, a salt with ammonium or an organic amine such as monoethanolamine, diethanolamine, and triethanolamine, a salt with a basic amino acid such as arginine, lysine, histidine, and ornithine, a salt with a nitrogen containing molecule belonging for example to the class of alkaloids or other nitrogen-containing natural product such as 1-methyl pyridinium or trigonellin.

Quinic acid may be obtained from a wide range of natural sources.

Examples of sources which contain relatively high amounts of quinic acid include sea buckthorn, bilberry, whortleberry, blueberry, kiwifruit, tamarillo, prune, crowberry, cranberry, peach, apple, sunflower, coffee, mayhaw and tea.

In one embodiment, the quinic acid is obtained from roasted coffee.

Examples of sources which contain intermediate amounts of quinic acid include grapes, black current, medlar, babaco, blackberry, bartlett pear, orange, lemon, citrus, cocoa, quince, chicory, feijoa, pear, sweet potato, japanese persimmon, tomato and banana.

Examples of sources which contain a low amount of quinic acid include pineapple, olive, cherry, pepino, prickly pear, fenugreek, bitter melon and red chicory.

Quinic acid may also be produced synthetically, for example by hydrolysis of chlorogenic acid and other quinic acid containing esters.

In certain embodiments, the present invention does not include the use of quinic acid in combination with a free branched chain amino acid and/or a metabolite thereof. In one embodiment the branched chain amino acid and/or a metabolite thereof may be leucine, valine, isoleucine, 4-hydroxyisoleucine, keto-isocaproic acid (KIC), alpha-hydroxy-isocaproic acid or β-hydroxy-β-methylbutyrate (HMB).

In one embodiment, the present invention does not include that use of quinic acid in combination with leucine and/or a metabolite thereof.

In one embodiment, the present invention does not include the use of quinic acid in combination with HMB.

Promoting Energy Expenditure

Energy expenditure refers to the process of metabolising energy sources in biological tissues and the conversion into chemical energy or heat energy. Energy expenditure may refer to the amount of physiochemical energy produced during the process.

Promoting energy expenditure may refer to promoting the amount of physiochemical energy produced by an individual.

The amount of energy expended may be calculated from the amount of oxygen consumed. Energy expenditure may also be determined by measuring the amount of carbon dioxide discharged and the amount of oxygen consumed by an individual (e.g. by measuring the amount of oxygen and carbon dioxide in a subject's breath). The ratio between the amount of carbon dioxide produced and the amount of oxygen consumed in one breath is known as the respiratory exchange ratio (RER). Measuring this ratio can be used for estimating the respiratory quotient (RQ), which is a function of the amount of carbon dioxide discharged/the amount of oxygen consumed by an individual. RQ is an indicator of whether carbohydrate or fat is being metabolized to supply the body with energy.

Calculating RER and RQ values is standard practice in the art, for example see Simonson & DeFonzo (Am. J. Physiol. 1990; 258:E399-12) and Pendergast et al. (J. Am. Coll Nutr. 2000; 19:345-50).

The amount carbon dioxide consumed and oxygen discharged by an individual may be determined using methods and apparatus which are well known in the art. For example, the percentages of oxygen and carbon dioxide in inspired and expired air may be measured using standard gas analysers.

Promoting energy expenditure includes promoting carbohydrate burning and/or fat burning.

Carbohydrate burning means that carbohydrate is metabolized in biological tissues and converted into chemical energy or heat energy. The amount of carbohydrate burned may be calculated from the amount of oxygen consumed and the amount of carbon dioxide discharged during that process by using, for example, the following equation described by Peronnet et al. (Can. J. Sport. Sci., 1991, vol. 16, 23-29):

The amount of carbohydrate burned=(4.585×RQ−3.226)×the amount of oxygen consumed.

This value indicates the amount of carbohydrate-derived energy produced by the individual. Promoting carbohydrate burning may refer to increasing the amount of carbohydrate burned as defined above.

The compound for use according to the first aspect of the present invention may be for use in promoting fat burning.

Fat burning means that fatty acids are metabolized in biological tissue and converted into chemical energy or heat energy. The amount of fat burned is calculated from the amount of oxygen consumed and the amount of carbon dioxide discharged during the oxidative metabolism process of fat by using, for example, the following equation of Peronnet, et al. (as above):

The amount of fat burned=1.695×(1−1.701/1.695×RQ)×the amount of oxygen consumed.

This value indicates the amount of fat-derived energy produced at an individual level. Promoting fat burning may refer to increasing the amount of fat burned as defined above.

Thermogenesis

Thermogenesis is a component of the metabolic rate. It refers to the process of heat production in organisms, particularly in warm-blooded animals. Thermogenic methods may be classified as Exercise-associated thermogenesis (EAT), Non-exercise activity thermogenesis (NEAT) or diet-induced thermogenesis.

Thermogenesis may be achieved by physical shivering or by non-shivering mechanisms. Non-shivering thermogenesis may occur in brown adipose tissue through a mechanism which involves uncoupling protein-1, which allows the uncoupling of protons moving down their mitochondrial gradient from the synthesis of ATP, thus allowing the energy to be dissipated as heat (Cannon et al.; 2004; Phys. Rev; 84(1); 277-359). Thermogenesis is thus a mechanism of energy expenditure.

Various substances are known to promote thermogenesis. Examples of such substances include caffeine, ephedrine, ephedra, bitter orange, capsicum, ginger, guar gum and pyruvate.

The present inventors have determined that quinic acid is a thermogenic substance. Thus the compound for use as described herein may be for use in promoting thermogenesis.

Maintaining body temperature refers to the ability of a subject to keep its body temperature within certain boundaries when the surrounding temperature is very different. For example, the average oral temperature for a healthy human adult is typically between 36.3-37.3° C. As an example, maintaining body temperature may refer to maintaining body temperature when the temperature of the external environment is lower than the optimal body temperature of the subject.

The present inventors have determined that administration of quinic acid improves the ability of a subject to maintain its body temperature in a low temperature environment.

Thus the compound for use as described herein may be for use in maintain body temperature in a subject.

Weight Loss

Attaining weight loss as defined herein may refer to a reduction in parameters such as weight (e.g. in kilograms), body mass index (kgm⁻²), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention from the value of said parameter at the onset of the intervention (e.g. a use according to the present invention).

The degree of weight loss may be expressed as a percentage change of one of the aforementioned weight phenotype parameters (e.g. a percentage change in a subject's body weight (e.g. in kilograms) or body mass index (kgm²). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in risk for obesity related co-morbidities.

Maintaining weight loss as defined herein may refer to the maintenance in parameters such as weight (e.g. in kilograms), body mass index (kgm⁻²), waist-hip ratio (e.g. in centimetres) fat mass (e.g. in kilograms), hip circumference (e.g. in centimetres) or waist circumference (e.g. in centimetres) following an intervention.

Typically, maintenance of weight loss occurs after a period of attaining weight loss.

In one aspect, the present invention provides the non-therapeutic use of formula 1 or 1a to maintain a healthy body composition after a period weight loss.

The degree of weight maintenance may be calculated by determining the change in one or more of the aforementioned parameters during a period of time. The period of time may be for example at least 12, 15, 20, 26, 30, 36, 40, 46 or 50 weeks.

The degree of weight maintenance may be expressed as the weight regained during a period following attainment of weight loss, for example as a percentage of the weight lost during attainment of weight loss.

In one aspect, the present invention provides a compound of structural formula 1 or 1a for use in attaining or maintaining weight loss in a subject.

Obesity “Overweight” is defined for an adult human as having a BMI between 25 and 30. “Body mass index” or “BMI” means the ratio of weight in kg divided by the height in metres, squared. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30. “Normal weight” for an adult human is defined as a BMI of 18.5 to 25, whereas “underweight” may be defined as a BMI of less than 18.5.

Obesity is a chronic metabolic disorder that has reached epidemic proportions in many areas of the world and is the major risk factor for serious co-morbidities such as type 2 diabetes mellitus, cardiovascular disease, dyslipidaemia and certain types of cancer (World Health Organ Tech Rep Ser. 2000; 894:i-xii, 1-253).

Obesity related disorder refers to any condition which an obese individual is at an increased risk of developing.

The obesity-related disorder may be diabetes (e.g. type 2 diabetes), stroke, high cholesterol, cardiovascular disease, insulin resistance, coronary heart disease, metabolic syndrome, hypertension or fatty liver.

In one embodiment the obesity-related disorder is not diabetes.

In one embodiment the obesity-related disorder is not insulin resistance.

In one aspect, the present invention provides a compound of structural formula 1 or 1a for use in the treatment or prevention of obesity or an obesity related disorder.

The compound for use as described herein may be for use in reducing fat mass and substantially maintaining lean mass in a subject.

Fat mass refers to the portion of a subject's body which is composed of fat. Fat mass may be determined using a wide range of methods, for example caliper-based measurements of skinfold thickness, Dual energy X-ray absorptiometry, CT or MRI scanning or bioelectrical impedance analysis.

Reducing fat mass may mean that fat mass is reduced by at least 1%, at least 2%, at least 5%, at least 10%, at least 15%, at least 20%, at least 30%, at least 40% or at least 50%.

Maintaining lean body mass is important for optimal metabolism, normal physical activity and good health.

Substantially maintaining lean mass may mean that lean mass alters by, for example, less than 7%, less than 5%, less than 4%, less than 3%, less than 2% or less than 1% following or during an intervention.

Preferably the majority of weight loss is due to a reduction in non-lean mass rather than lean mass.

Stamina

Stamina refers to the ability of an individual to sustain physical effort over a prolonged period.

Improving stamina in a subject may refer to increasing the length of time for which a subject can perform a physical activity at a given level. Improving stamina in a subject may mean to increase the performance achieved by a subject in a physical or mental activity in a given period of time.

In one aspect the present invention relates to the use of a compound of structural formula 1 or 1a for improving stamina in a subject.

Exercise Effect

Improving an exercise effect refers to enhancing favourable effects inherently obtained by exercise compared to when the exercise is performed alone. In other words, improving an exercise effect means that a favourable exercise effect is augmented by the use of a compound according to structural formula 1 or 1a.

The favourable exercise effect may be, for example, promoting weight loss as a result of exercise, promoting energy expenditure, promoting carbohydrate burning, promoting fat burning and/or an anti-obesity effect.

Composition

The present invention further provides a composition comprising a compound of formula 1 or 1a as an active agent for use as described herein.

The composition may be selected from the group consisting of a food product, a food extract, drink, food additive, nutritional supplement, medical food, pet food product and a powdered nutritional formulation to be reconstituted in milk or water.

In general terms, administration of the composition may be by oral route.

In one embodiment the compound of formula 1 or 1a is administered in a pharmaceutical or nutraceutical composition. The pharmaceutical composition comprises the compound of formula 1 or 1a and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals.

Enriched

In one aspect the present invention provides a composition, a food or food extract which is enriched with a compound of formula 1 or 1a.

The terms “food” or “food extract” or “food composition” or “food product” means a product or composition that is intended for ingestion by an animal, including a human, and provides nutrition to the animal.

‘Enriched’ means that a compound of formula 1 or 1a has been added to the food or food product. For example, the compound may be spiked (i.e. added within or into) the food or food extract.

In one embodiment, where a food or food extract natively contains quinic acid, enriched with a compound of formula 1 or 1a means that the enriched food or food extract comprises a greater amount of the compound than occurs natively in the food or food extract.

For example, an enriched composition, food or food extract may comprise at least 1.5-, at least 2-, at least 5-, at least 10-, at least 20-, at least 50- or at least 100-fold more quinic acid than an equivalent native composition, food or food extract which has not been enriched.

Enrichment may be achieved by adding a compound of formula 1 or 1a to the composition, food or food extract, for example by spraying or spiking it with the compound.

The composition, food or food extract may comprise a compound of formula 1 or 1a at a concentration of at least 0.5, at least 1, at least 2, at least 5, at least 10, at least 20, at least 50 or at least 100 g/kg.

The food extract may be derived from sea buckthorn, bilberry, whortleberry, blueberry, kiwifruit, tamarillo, prune, crowberry, cranberry, peach, apple, sunflower, coffee, mayhaw, tea, grapes, black current, medlar, babaco, blackberry, bartlett pear, orange, lemon, citrus, cocoa, quince, chicory, feijoa, pear, sweet potato, japanese persimmon, tomato and banana, pineapple, olive, cherry, pepino, prickly pear, fenugreek, bitter melon or red chicory.

The food extract may be derived from a source which contains high levels of quinic acid, for example sea buckthorn, bilberry, whortleberry, blueberry, kiwifruit, tamarillo, prune, crowberry, cranberry, peach, apple, sunflower, coffee, mayhaw or tea.

The food extract may be derived from coffee, tea, bilberry, kiwi fruit sea buckthorn, tamarillo, prune, cranberry, peach, apple or crowberry.

The food extract may be derived from a fermented product, for example vinegar or tea.

Diet Product

As used herein, a diet product may be a meal replacement product or a supplement product. The diet product may, for example, suppress the subject's appetite. The diet product can include food products, drinks, pet food products, food supplements, nutraceuticals, food additives or nutritional formulas.

Diet products may be in any form, e.g. solid, liquid, gel, tablet, capsule, powder, and the like. The dietary product can have any suitable form such as a gravy, drinking water, beverage, yogurt, powder, granule, paste, suspension, chew, morsel, treat, snack, pellet, pill, capsule, tablet, sachet, or any other suitable delivery form. Preferably they are provided in convenient dosage forms, e.g. in sachets. Dietary products can be provided in bulk consumer packages such as bulk powders, liquids, gels, or oils. In soft capsules, the active ingredients are preferably dissolved or suspended in suitable liquids, such as fatty oils, paraffin oil or liquid polyethylene glycols. Optionally, stabilizers may be added. Diet products can be provided in bulk quantities to be included in other food items such as snacks, treats, supplement bars, beverages, and the like.

The diet product can comprise optional compounds such as vitamins, preservatives, probiotics, prebiotics, and antioxidants. The product may be administered in small amounts, or in the alternative, can be diluted before administration. The diet product may require admixing with a food composition or with water or other diluent prior to administration.

In one embodiment, the diet product may comprise a product such as Optifast® or Modifast®.

In another embodiment, the diet product may comprise, for example, a composition which is 46.4% carbohydrate, 32.5% protein and 20.1% with fat, vitamins, minerals and trace elements; 2.1 MJ per day (510 kcal/day).

Use

The present invention provides the use of a compound of formula 1 or 1a in the preparation of a product for promoting energy expenditure, promoting thermogenesis, improving stamina and/or improving an exercise effect.

The product may be any product as described herein, for example a composition or diet product as described herein.

The present invention also provides use of a compound of formula 1 or 1a in the preparation of a diet product as defined herein.

Method

In one aspect the present invention relates to a method for promoting energy expenditure, promoting thermogenesis, improving stamina and/or improving an exercise effect comprising administering compound of formula 1 or 1a to a subject.

The compound may be administered in the form of a composition or product as described herein.

The method comprises administering an effective amount of compound of formula 1 or 1a to the subject. An effective amount refers to an amount which is capable of, for example, attaining or maintaining weight loss, treating or preventing obesity or an obesity-related disorder, promoting energy expenditure, promoting thermogenesis, improving stamina and/or improving an exercise effect.

The effective amount may differ depending on the weight, age or sex of the subject.

Subject

The subject may be, but is not limited to, mammals such as bovine, canine, caprine, cervine, equine, feline, human, ovine, porcine and primates. The subject may be a companion animal. The subject may be a human.

In various embodiments, the subject may have, or be suspected of or at risk of, obesity or an obesity related disorder.

Those skilled in the art will understand that they can freely combine all features of the present invention described herein, without departing from the scope of the invention as disclosed.

Various preferred features and embodiments of the present invention will now be described by way of non-limiting examples.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

### EXAMPLES

### Example 1—Quinic Acid is Tolerated at Both Low and High Doses in Mice Fed on a Chow Diet and Mice Fed on a High Fat Diet

Mice were fed a low fat diet supplemented with either water (Veh) or quinic acid at low dose (25 mg/(kg*day)) or high dose (HD, 100 mg/(kg*day). Food intake was calculated between 12 and 18 weeks of age by weighing the food placed on the tray of the cage at the start of the week and the amount remaining 5 days later.

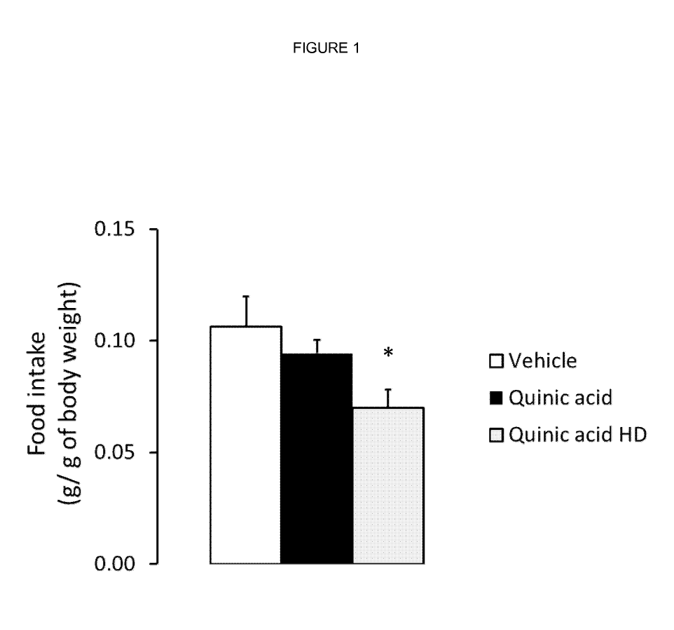

Quinic acid at high dose caused food aversion in mice on chow diet (FIG. 1).

Mice were fed a high fat diet (60% fat) supplemented with either water (Veh) or quinic acid at low dose (25 mg/(kg*day)) or high dose (HD, 100 mg/(kg*day). Calculations for food intake were made in the same manner as for mice on chow diet.

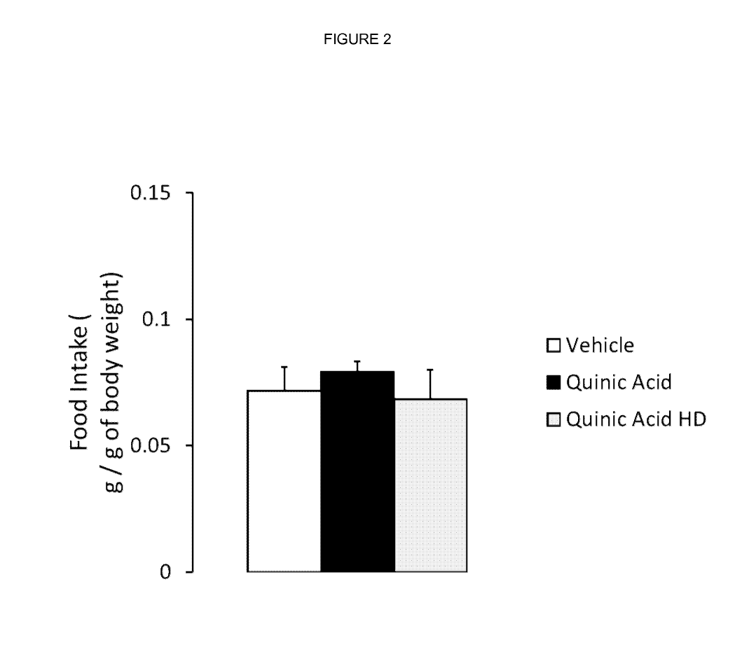

Quinic acid did not cause food aversion in mice on high fat diet (FIG. 2).

### Example 2—Quinic Acid Prevents Body Weight Gain in Low- and High-Fat Diets

Body weight evolution on mice fed on chow diet or high-fat diet, supplemented with either water (Veh) or Quinic Acid (25 mg/(kg*day) was calculated.

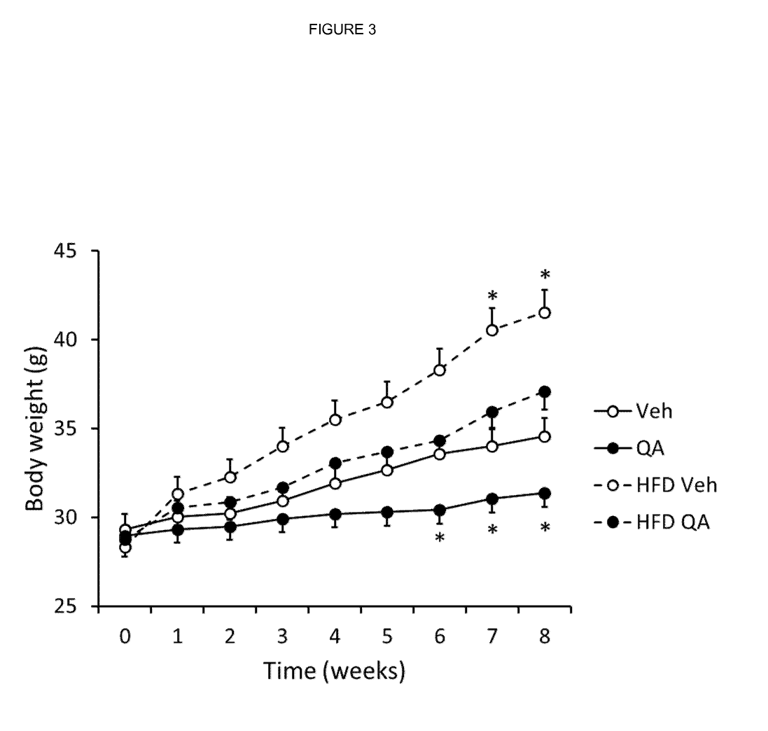

Quinic acid reduced weight gain in mice on either low or high-fat diet (FIG. 3).

### Example 3—Quinic Acid Prevents Fatty Acid Deposition

Body lean and fat mass composition of 18 week-old mice fed on chow diet or high-fat diet, supplemented with either water (Veh) or Quinic Acid (25 mg/(kg*day) was measured by nuclear magnetic resonance using an Echo-Magnetic Resonance Imaging (Echo-MRI) system (Echo Medical System LCC).

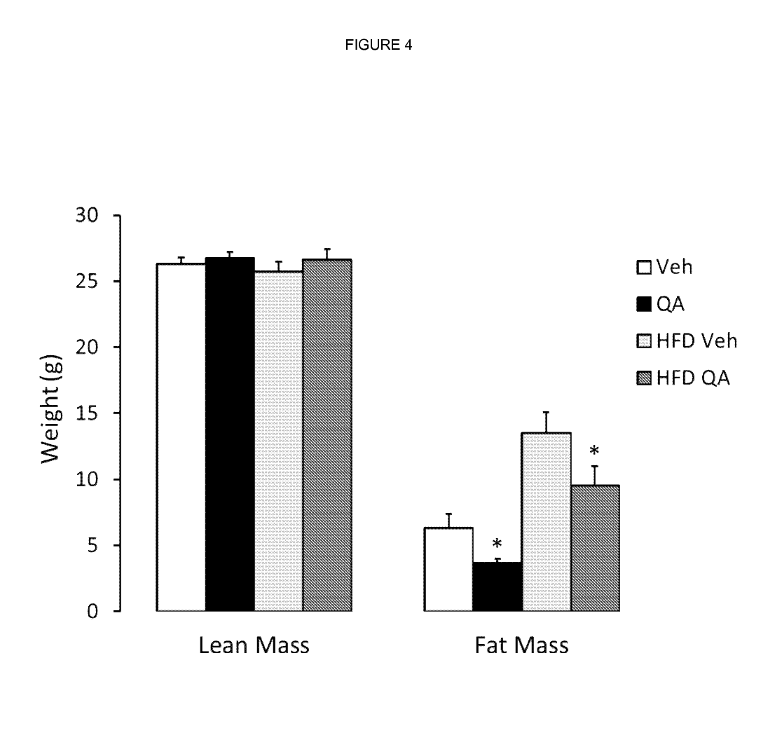

Quinic acid reduced fat deposition in mice on chow diet and in mice on high-fat diet (FIG. 4).

### Example 4—Quinic Acid does not Alter Daily Activity in Mice

18 week-old mice were placed on a comprehensive laboratory animal monitoring system (CLAMS; Columbus Instruments Inc.) for 24 hrs after a 24 hr habituation to CLAMS cages. Total ambulatory activity of mice fed on chow diet, supplemented with either water (Veh) or Quinic Acid (25 mg/(kg*day) was measured by examining how many times mice crossed diverse laser beams in the cage, as instructed by the manufacturer.

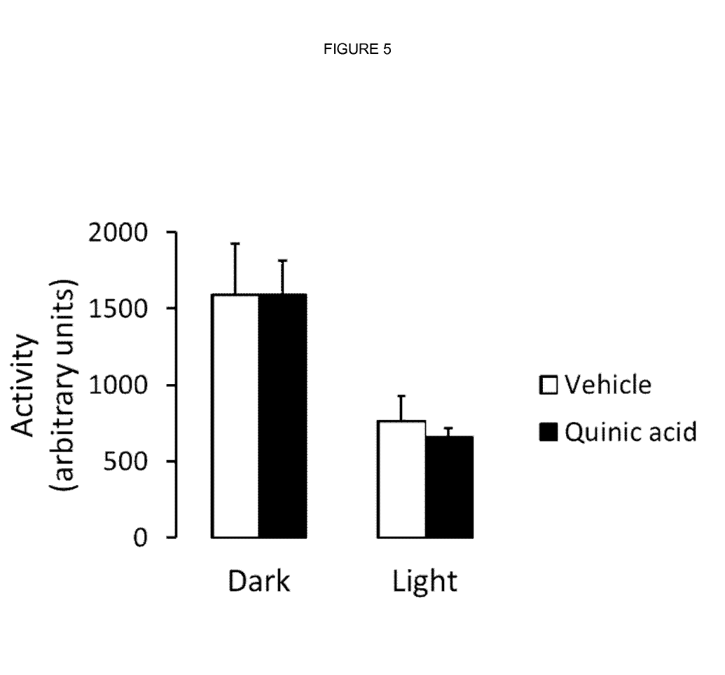

Quinic acid did not alter daily activity in mice (FIG. 5).

### Example 5—Quinic Acid Enhances Energy Consumption and Carbohydrate Metabolism During the Dark Phase

Oxygen consumption and respiratory exchange ratio (RER) of 18 week-old mice fed on chow diet, supplemented with either water (Veh) or Quinic Acid (25 mg/(kg*day), was measured using a CLAMS system during 24 hrs after 24 hrs of habituation of mice to CLAMS cages.

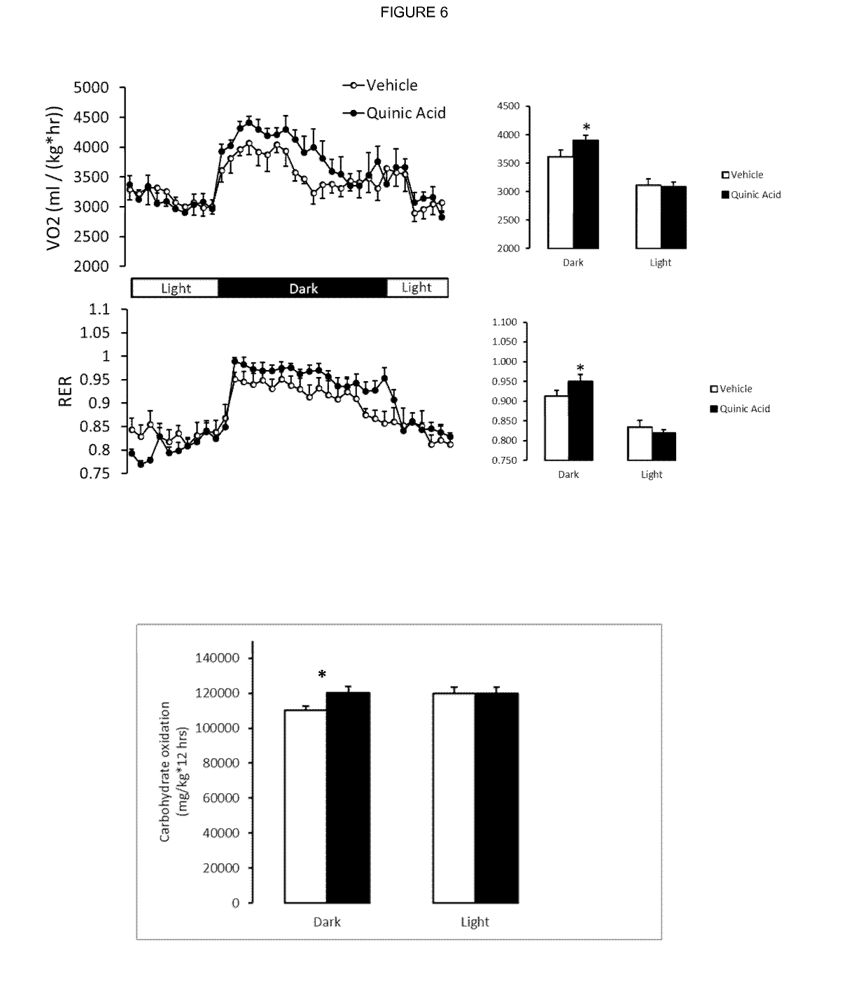

Quinic acid enhanced energy consumption and carbohydrate metabolism during the dark phase (FIG. 6). The total carbohydrate oxidation was calculated based using the equation: carbohydrate oxidation=(4.55*V02-3.21*VCO2) where VO2 is volume of oxygen consumed and VCO2 is volume of CO2 produced. These equations are based on Even and Nadkarny: “Indirect calorimetry in laboratory mice and rats: principles, practical considerations, interpretation and perspectives”; Am J Physiol Regul Integr Comp Physiol 303: R459-R476, 2012.

### Example 6—Quinic Acid Modestly Enhances Glucose Tolerance

Glucose excursion curves were determined after an intraperitoneal injection of glucose (2 g/kg) on 12 hr fasted 20 week-old mice fed a chow diet or a HFD, supplemented with either water (Veh) or Quinic Acid (25 mg/(kg*day).

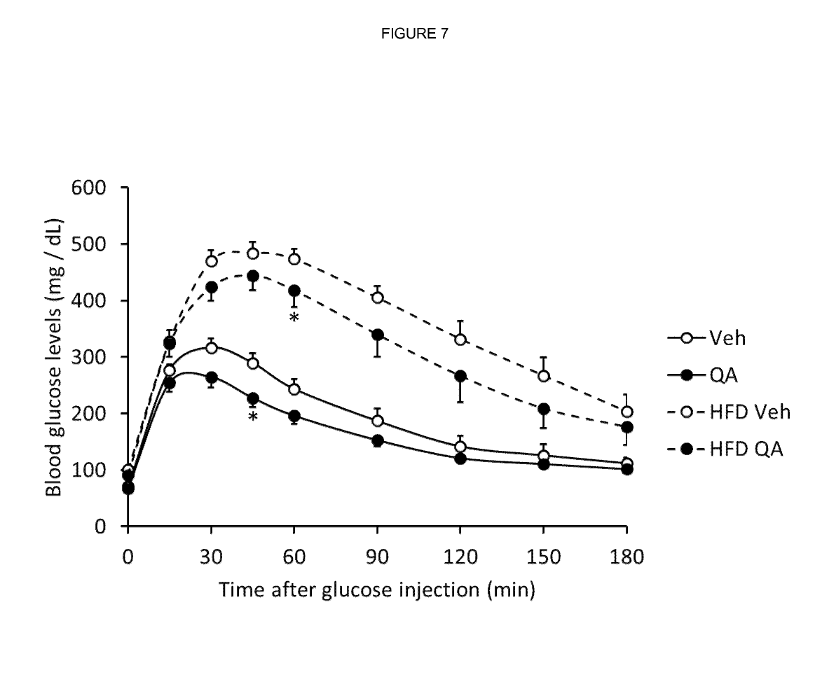

Quinic acid moderately enhanced glucose tolerance (FIG. 7).

### Example 7—Quinic Acid does not Alter Insulin Response in Chow-Fed Mice

Glucose excursion curves were determined after an intraperitoneal injection of insulin (0.3 U/Kg) after a 6 hr fast on 22 week-old mice fed a chow diet, supplemented with either water (Veh) or Quinic Acid (25 mg/(kg*day).

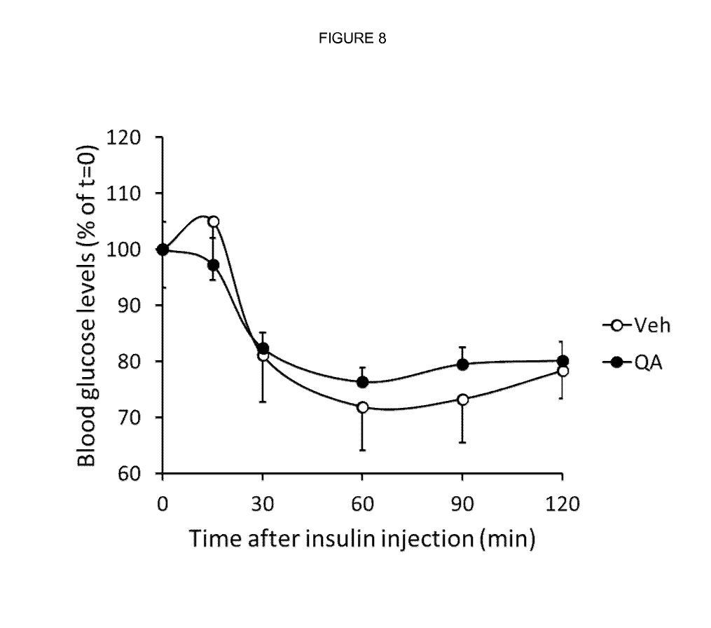

Quinic acid did not alter insulin response in chow-fed mice (FIG. 8).

### Example 8—Quinic Acid Lowers Basal Glycemia but does not Significantly Affect Insulin Response in High-Fat Fed Mice

Glucose excursion curves were determined after an intraperitoneal injection of insulin (0.75 U/Kg) after a 6 hr fast on 22 week-old mice fed a high-fat diet, supplemented with either water (Veh) or Quinic Acid (25 mg/(kg*day).

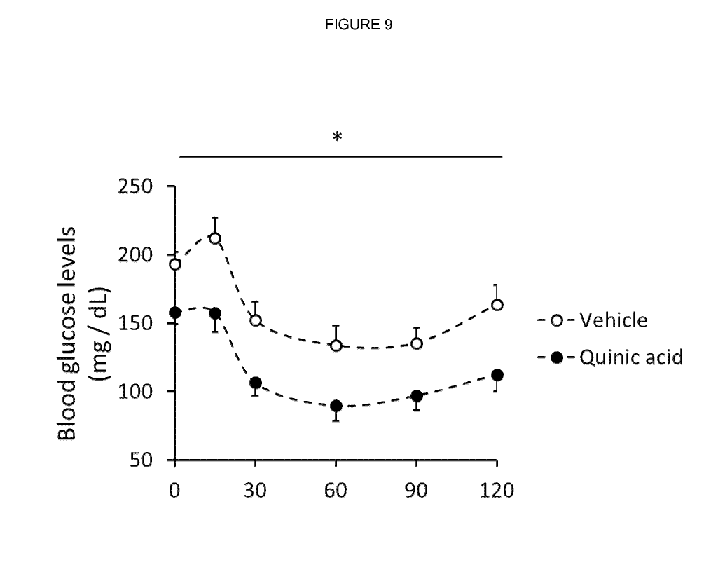

Quinic acid lowered basal glycemia but did not significantly affect insulin response in high-fat fed mice (FIG. 9).

### Example 9—Quinic Acid Enhances Thermogenic Function

Rectal temperature was measured at 8 a.m. (t=0) on 24-week old mice fed a chow diet, supplemented with either water (Veh) or Quinic Acid (25 mg/(kg*day). Then, mice were placed on a cold room (6° C.), with no access to food, and rectal temperature was measured every hour.

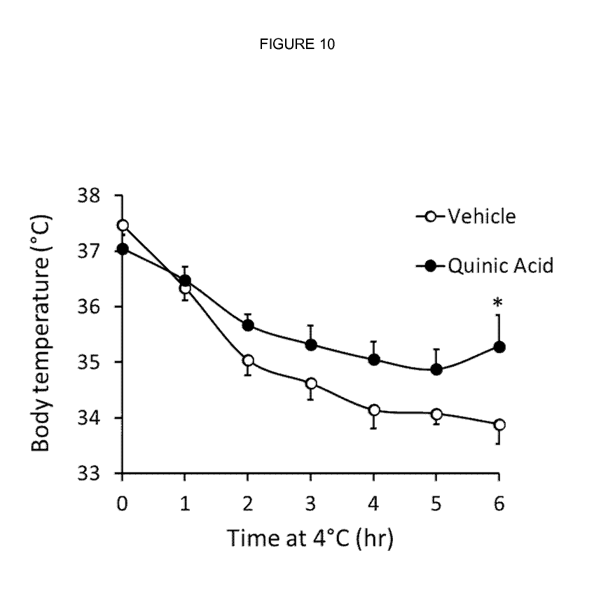

These data demonstrate that quinic acid enhanced thermogenic function (FIG. 10).

### Example 10—Quinic Acid Potentiates Endurance Performance on High-Fat Fed Mice

26 week-old mice were place on a treadmill and endurance was evaluated by using a increasing speed protocol, as described previously (Lagouge et al., 2006, PMID: 17112576). The tests were performed on low and high fat-fed mice supplemented with either water (Veh) or Quinic Acid (25 mg/(kg*day).

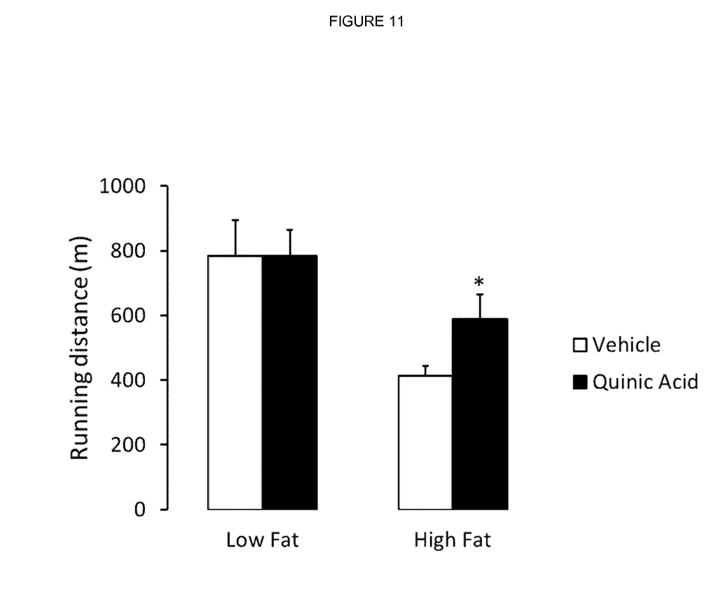

These data demonstrate that quinic acid potentiates endurance performance in high-fat fed mice (FIG. 11).

Materials and Methods

Food Preparation

Powder low fat diet (LFD; Research Diets Inc.; Ref: D12450J) was reconstituted into ˜20 gram pellets using 100 ml of water/kg of food. In addition to normal low fat diet pellets, two additional groups were made by adding quinic acid (QA) to the water used for reconstitution. A first group (low dose) was created, where 0.25 g of QA were added per Kg of food (this means, 0.25 grams per 100 ml of water). In a second group (high dose), 1 g of QA was added per Kg of food (this means, 1 gram per 200 ml of water). The low doses and high doses were considered as ˜25 mg/(kg*day) and ˜100 mg/(kg*day), respectively, assuming that mice averaged 30 grams at the beginning of the experiments and ate approximately 3 grams of food per day. Once pellets were made, they were left drying under a hood for 24 hrs, flipping the upside down after 12 hrs. Then, pellets were stored in a −20° C. freezer.

In the case of high-fat diet (HFD; Research Diets Inc.; Ref: D12492), the diet preparation was similar, but with two minor changes. First, 20 ml of water, instead of 100 ml, was used per Kg of food for pellet preparation. Second, the pellets were frozen 1 hr after preparation.

All pellets were thawed at room temperature at 8a.m prior to changing the food in the cages at 10a.m.

Treatment

At 10 weeks of age, 60 male mice were randomly distributed in 6 groups (n=10 per group) and provided the different diets: LFD (Control, Low Dose and High Dose) or HFD (Control, Low Dose and High Dose). Mice were maintained for 8 weeks on their corresponding diets before phenotyping took place. During this time, body weight and food intake was monitored weekly.

1. A compound of structural formula 1: 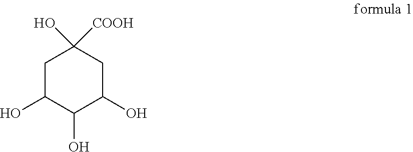 [SMILES: O=C(O)C1(O)CC(O)C(O)C(O)C1]
or a salt thereof for use in promoting energy expenditure and/or thermogenesis.

2. A compound according to claim 1 wherein the compound is for use in promoting carbohydrate burning.

3. A compound according to claim 1 wherein the compound is for use in attaining or maintaining weight loss in a subject.

4. A compound according to claim 1 wherein the compound is for use in the treatment or prevention of obesity or an obesity related disorder.

5. A compound according to claim 3 wherein the compound is used to reduce fat mass and substantially maintain lean mass.

6. A compound according to claim 1 wherein the compound is for use in maintaining body temperature in a subject.

7. A compound according to claim 1 for use in improving stamina in a subject.

8. A compound according to claim 1 for use in improving an exercise effect.

9. A compound for use according to claim 1 wherein the compound has the structural formula 1a:

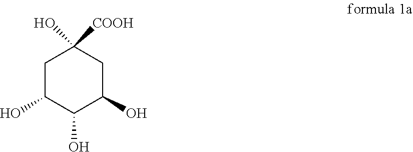 [SMILES: O=C(O)[C@]1(O)C[C@@H](O)[C@@H](O)[C@H](O)C1]

10. A composition comprising a compound of formula 1 or 1a as an active agent for use in accordance with any preceding claim.

11. A composition according to claim 10 wherein the composition is selected from the group consisting of a food product, a food extract, drink, food additive, nutritional supplement, medical food, pet food product and a powdered nutritional formulation to be reconstituted in milk or water.

12. A food or food extract enriched with a compound of formula 1 or 1a.

13. A food or food extract according to claim 11 wherein the food extract is derived from coffee, sea buckthorn, bilberry, whortleberry, blueberry, kiwifruit, tamarillo, prune, crowberry, cranberry, peach, apple, sunflower, mayhaw, tea, grapes, black current, medlar, babaco, blackberry, bartlett pear, orange, lemon, citrus, cocoa, quince, chicory, feijoa, pear, sweet potato, japanese persimmon, tomato, banana, pineapple, olive, cherry, pepino, prickly pear, fenugreek, bitter melon or red chicory.

14. A food or food extract according to claim 13 wherein the food extract is derived from a fermented product.

15. A food or food extract according to claim 14 wherein the fermented product is vinegar or fermented tea.

16. A pharmaceutical or nutraceutical composition comprising a compound of formula 1 or 1a.

17. A diet product for use as part of a low calorie diet for weight loss, wherein the diet product comprises a compound of formula 1 or 1a.

18. Use of a compound of formula 1 or 1a in the preparation of a product for promoting energy expenditure, promoting thermogenesis, improving stamina and/or improving an exercise effect.

19. Use of a compound of formula 1 or 1a in the preparation of a diet product.

20. A method for promoting energy expenditure, promoting thermogenesis, improving stamina and/or improving an exercise effect comprising administering compound of formula 1 or 1a to a subject.
